(19) United States
(12) Patent Application Publication
(10) Pub. No.: US 2008/0308108 A1
(43) Pub. Date: Dec. 18, 2008

(54) Oral cannula

(76) Inventor: Melanie Paige Diorio, Maumelle, AR (US)

Correspondence Address: Kyla D. Cummings;SPEED LAW FIRM, Suite 1200, 111 Center Street, Little Rock, AR, 72201, US

(21) Appl. No.: 11/818,424

(22) Filed: Jun. 14, 2007

(51) Int. Cl. A61M 16/00

(52) U.S. Cl. 128/207.14; 128/207.18

(57) ABSTRACT

Embodiments include a method and apparatus for maintaining an open airway passage in a patient while having the capacity to provide supplemental gas such as oxygen to the patient. In one embodiment, the apparatus includes an oral cannula comprising an extended cannula member and an oral airway member. The method may include inserting the oral cannula into a pharynx of the patient and providing gas such as oxygen directly to an opening of a glottis of the patient. Other embodiments include a kit comprising a nasal cannula member comprising a delivery cannula and at least two cannula prongs, an oral airway member, and at least two cannula extension members, each cannula extension member connectable to each nasal cannula member prong.

### BACKGROUND OF THE INVENTION

1. Field of the Invention

Embodiments generally relate to an apparatus and method for use in anesthesia or sedation.

2. Description of the Related Art

General anesthesia and monitored anesthesia care (MAC) include the use of sedatives and other agents introduced mainly intravenously or as inhalants. General anesthesia is a deep state of sleep where the patient loses consciousness and sensation and requires assisted ventilation.

The MAC type of anesthesia is routinely performed in hospitals all over the United States to provide safer sedation for patients undergoing uncomfortable procedures and/or minor surgeries. MAC technique includes the use of intravenous drugs to provide anxiolysis, analgesia, and/or amnesia to a patient undergoing procedures which would otherwise be unacceptably uncomfortable to the patient. MAC provides the patient with anxiety relief, amnesia, pain relief, comfort, and safety during procedures. Like general anesthesia, MAC involves using sedatives and other agents, but the dosage is low enough that patients typically remain responsive and breathe without assistance, so that the patients generally do not require assisted ventilation. MAC may be used to supplement local or regional anesthesia (which involve providing numbness to a local or regional area of the patient's body). During MAC, the patient is sedated and amnestic, but usually remains responsive, and is in a light sleep and may or may not wake up from time to time during the procedure. MAC is especially useful to provide anesthesia to sick and elderly patients who often cannot physically handle the stronger drugs, deep sleep inducement, or machine-assisted breathing of general anesthesia.

The general MAC procedure is as follows. An intravenous line (IV) through which medications may be administered to the patient is placed in the patient. A sedative is administered to the patient through the IV. Supplemental oxygen may be given to the patient if needed. Anesthesia medications may include sedatives for anxiety relief and amnesia, opioids and local anesthetic for pain relief, and/or antiemetics for treating nausea and vomiting. Other medications needed to treat any existing medical condition such as diabetes, high blood pressure, etc. may also be given to the patient.

Although the MAC technique is often advantageous over general anesthesia due to its relative safety, the incidence of obesity in our country (and abroad) has made this choice less desirable as it is currently performed. An estimated thirty percent of adults in the United States weigh more than twenty percent above what is considered the optimum body weight for their height. In patients who fall into this category, oxygenation becomes of heightened concern to the anesthesia provider due to the increased difficulty of managing the patient's airway to keep it unobstructed during sedation. The anatomic changes that occur in the overweight or obese patient include fleshy cheeks, a large tongue, and copious flaps of palatal pharyngeal and supralaryngeal soft tissue, one or all of which often obstruct the airway when a patient is sedated (a tongue falling back into the airway is referred to as “obstructed”). Other circumstances in which oxygenation and managing the patient's airway to avoid its obstruction are of particular concern are when the patient suffers from sleep apnea or when the procedure being performed involves an operation on the nose (such as skin cancer removal from the nose).

To provide adequate ventilation or oxygenation during MAC, supplemental oxygen may need to be delivered to the patient or the airway may need to opened or maintained, or both. An oral airway device may be used to open and maintain an open airway. An oral airway device is placed in a patient's mouth to keep the tongue and other obstructions from preventing adequate ventilation or oxygenation through the oral airway. A nasal cannula device may be used to provide supplemental oxygenation through the nasal passages of the patient via supplemental oxygen delivery through the cannula.

Upon sedation of the patient, it is often necessary to perform both the functions of maintaining and opening the oral airway passageway of the patient as well as providing supplemental ventilation or oxygenation or other gases to the patient. Inserting the oral airway device into the patient's oral airway passageway to open or maintain the open oral airway passageway and inserting the nasal cannula into the nasal airway of the patient is time-consuming and inconvenient because two separate motions with two separate devices must be employed to accomplish these tasks when time is often of the essence to ensure that a patient's well-being is not jeopardized due to improper ventilation. There is therefore a need for an efficient, time-saving, and convenient device and method which allow for proper ventilation and oxygenation of a patient under sedation. There is also a need for a device capable of and a method for accomplishing both tasks simultaneously, efficiently, and effectively.

### SUMMARY OF THE INVENTION

It is therefore an object of the present invention to provide an apparatus which permits timely, efficient, and convenient maintaining and/or opening of the oral airway passageway of a patient while allowing efficient delivery of supplemental gases (such as supplemental oxygen) to be introduced to the oral airway of the patient with the same apparatus.

It is a further object to provide an apparatus which permits maintaining and/or opening the oral airway passageway of a patient and allows supplemental gas delivery to a patient in one motion.

It is yet a further object to provide an efficient, time-saving, and convenient device and method which allows for proper ventilation and oxygenation of a patient under sedation or other anesthesia.

It is yet a further object to provide a device capable of and a method for efficiently and effectively accomplishing both proper ventilation and oxygenation of a patient simultaneously.

Yet further, an object of the present invention is to provide an oral cannula kit and method for assembling, where the components of the kit are capable of multiple configurations and uses.

Embodiments generally include an oral cannula apparatus comprising an oral airway member comprising an enlarged proximal end to prevent the proximal end from slipping down a patient's throat, the proximal end including a first passageway and a second passageway adapted to receive a cannula member therein, the first and second passageways extending along a length of the oral airway member, and a distal end, the distal end capable of leading the oral airway member upon its insertion into a mouth and pharynx of a patient; a cannula member having a first cannula and a second cannula extending from a delivery cannula, wherein the first cannula is located within the first passageway and the second cannula is located within the second passageway, the first and second cannula extending to a location at or near the distal end, and wherein the first and second cannula provide a delivery path therethrough to provide one or more gases from the first and second cannula directly to an opening of a glottis of the patient.

Embodiments also include a kit comprising a nasal cannula member comprising a delivery cannula and at least two cannula prongs extending generally perpendicularly from the delivery cannula; an oral airway member; and at least two cannula extension members, each cannula extension member connectable to each nasal cannula member prong.

Finally, embodiments also generally include a method comprising providing an oral cannula comprising an oral airway member having an enlarged proximal end and a distal end and first and second passageways extending from the proximal end to the distal end along the length of the oral airway member, and a cannula member having a first cannula and a second cannula extending generally perpendicularly from a delivery cannula, wherein the first cannula is located within the first passageway and the second cannula is located within the second passageway and the first and second cannula extend from the proximal end to a location at or near the distal end; and inserting the distal end of the oral cannula into the mouth and pharynx of a patient until the first and second cannula at the distal end provide direct gas flow access to an opening of a glottis of the patient.

### BRIEF DESCRIPTION OF THE DRAWINGS

So that the manner in which the above-recited features of embodiments of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a side sectional view of a cannula member and a proximal end sectional view of an oral airway member.

FIG. 2 is side sectional view of an oral cannula.

FIG. 3 is a perspective view of an underside of the oral cannula of FIG. 2.

FIG. 4 shows the oral cannula positioned in the pharynx of a patient.

FIG. 5 is a sectional view of portions of an oral cannula kit.

### DETAILED DESCRIPTION

Embodiments include an oral cannula device which is capable of maintaining an oral airway passageway of a patient open and free from obstruction while allowing supplemental oxygen to be provided to the patient efficiently and effectively through the oral cannula device. Embodiments include an oral cannula device capable of simultaneously performing both of these functions of maintaining an unobstructed oral airway and acting as a delivery device for supplemental oxygen provision through the patient's oral airway passage.

Embodiments also include an oral cannula device which is capable of directly, efficiently, and effectively providing supplemental oxygen to the posterior pharynx or the opening of the glottis of the patient while simultaneously maintaining an unobstructed oral airway passage in the patient. In some embodiments, this oral cannula device includes dual cannula extending on opposite sides of an oral airway device to provide oxygen to the patient at a distal end of the oral cannula device (the distal end being the end nearest the patient's posterior pharynx and the proximal end being the end nearest the oxygen supply or nearest the lips of the patient).

In some embodiments, the oral cannula device includes an oral airway device and a nasal cannula device having at least two prongs, the nasal cannula prongs operatively connected to the oral airway device at its proximal end and extending to a location at or near its distal end to provide a passageway for direct delivery of oxygen to the patient's posterior pharynx or glottic opening. Providing a direct passageway for delivery of oxygen to the patient's posterior pharynx or glottic opening is advantageous because less oxygen need be utilized to provide adequate oxygenation to the patient while the open oral airway passage of the patient is properly maintained.

Embodiments also include a method of providing a dual-functioning apparatus for providing oxygen directly to the oral airway opening while maintaining an unobstructed oral airway passage. The method may further include locating the dual-functioning apparatus within the oral airway passageway of the patient and maintaining the unobstructed oral airway passage of the patient. Additionally, the method may include providing oxygen to the distal end of the apparatus directly to the patient's oral airway passage (e.g., at or near the posterior pharynx or glottic opening) while the apparatus is disposed within the patient's oral airway passage and maintaining the open oral airway passage.

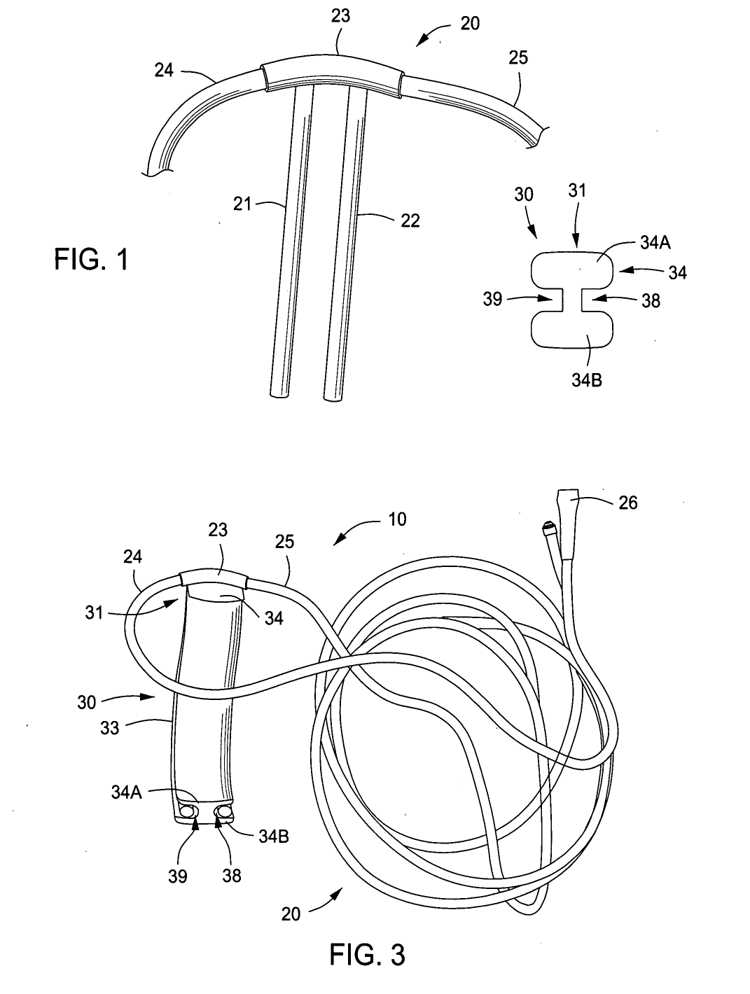

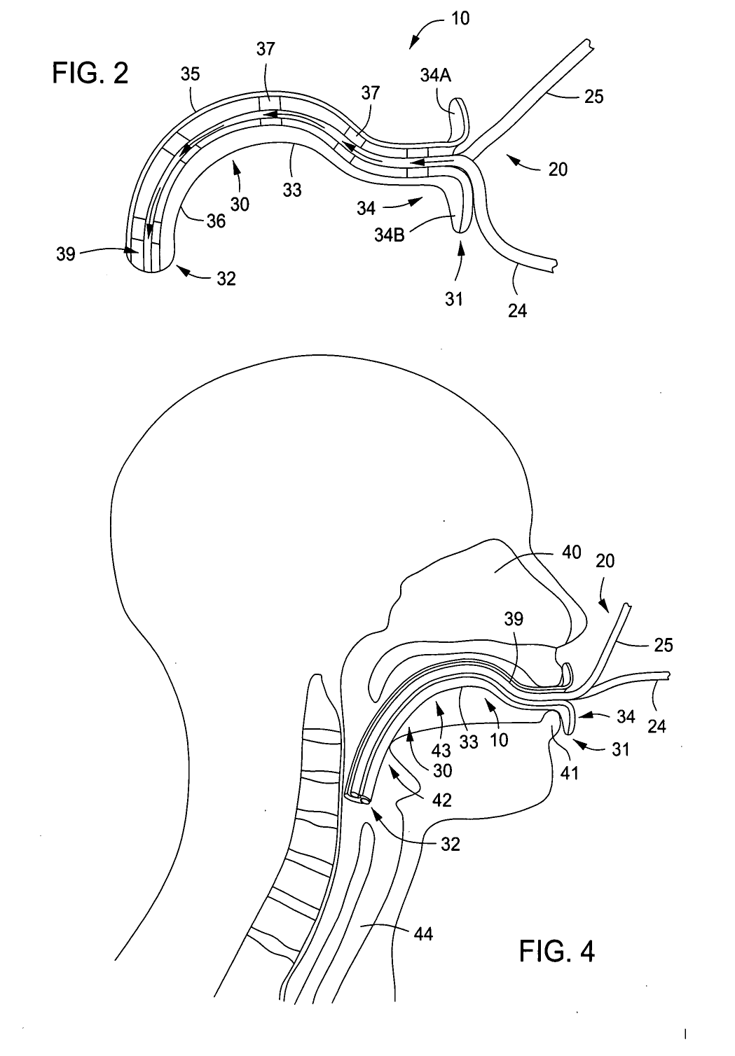

An oral cannula 10 is shown in FIGS. 2-4. The oral cannula 10 includes a nasal cannula member 20 and an oral airway member 30 (see FIG. 1) operatively connected to one another. The oral airway member 30 includes a body 33 having a proximal end 31 and a distal end 32. The body 33 is preferably curved so that the oral airway member 30 conforms to the curve of the throat and oral airway passage and over the tongue of the patient upon insertion of the distal end 32 into the patient's oral airway passage.

Although the oral airway member 30 may be made of a number of materials, in one example it may be constructed from a single, molded piece of hard plastic. Although the oral airway member 30 may be formed from a soft material and remain within the scope of embodiments of the present invention, it is preferred that the oral airway member 30 is constructed from a relatively hard and rigid material, such as hard plastic, so that the air passage through the oral airway member 30 is not obstructed if the patient bites down on the oral airway member 30 or otherwise exerts sufficient force on the oral airway member 30 to obstruct the air passage therethrough. In some embodiments, the oral airway member 30 is formed from a suitable rigid material, such as a relatively lightweight thermoplastic which may be injection molded into a shape, the injection molding method being known to those skilled in the art.

The body 33 of the oral airway member 30 includes two spaced apart plate members, including the upper plate member 35 and the lower plate member 36. The plate members 35 and 36 may be located generally parallel to one another and shaped generally the same. Extending between and connecting the plate members 35, 36 to one another are one or more connecting members 37, which may be spaced apart along the length of the plate members 35, 36.

On each side of the connecting members 37, a three-sided passageway is formed by an outward-facing surface of the connecting members 37, a portion of the inward-facing surface of the upper plate member 35, and a portion of the inward-facing surface of the lower plate member 36. These two passageways 38, 39 allow passage of air through the oral airway member 30, with or without the help of cannula (described below), while the oral cannula 10 maintains an open oral airway passageway in the patient.

The oral cannula 10 may include a flange 34 projecting upward and downward at or near its proximal end 31, which may be formed from a first flange portion 34A where the upper plate member 35 extends (e.g., curves) upward and from a second flange portion 34B where the lower plate member 36 extends (e.g., curves) downward. These flange portions 34A, 34B preferably cooperate with one another to form a shoulder against which a patient's lips may rest. A purpose of the flange portions 34A, 34B is to form a flange having a large enough outer diameter so that the oral airway member 30 cannot fall completely down into the patient's airway (therefore the flange portions 34A, 34B may cooperate with one another to form a flange with an outer diameter larger than a diameter of a patient's airway passage). Furthermore, another purpose of the flange portions 34A, 34B is to make the inner surface of the flange 34 capable of resting on and stopping the oral cannula at the patient's lips.

The oral cannula 10 further includes a cannula member 20. The cannula member 20 includes a first, hollow tubular member 21 and a second, hollow tubular member 22 which extend from a third, hollow tubular member 23. The first and second tubular members preferably are disposed generally perpendicular to the third tubular member 23, and gas is capable of flow between the first tubular member 21 and the third tubular member 23 as well as between the second tubular member 22 and the third tubular member 23. The third tubular member 23 is capable of receiving one or more tubes 24 and 25 therein to allow gas flow between the tubes 24 and 25 and the tubular member 23. The cannula member 20 may be formed in one piece or instead may be formed of multiple pieces which are connectable to one another.

The cannula member 20 may be used to provide one or more gases such as oxygen, air, and/or other gases to the patient through the oral airway member 30. The one or more gases may be introduced into the cannula member 20 by tubes 24 and/or 25. To accomplish this introduction of supplementary gas(es) into the tubes 24, 25, one or more ends 26 of the cannula member 20 may be connected to an oxygen supply.

The first and second tubular members 21 and 22 may be connected to the oral airway member 30 at one or more locations and extend along the length of the oral airway member 30 to provide a direct flow path for the one or more gases from the external gas source to the posterior pharynx or opening of the glottis. Preferably, the first and second tubular members 21, 22 both extend up to or near the distal end 32 of the oral airway member 30, but it is within the scope of embodiments to extend the tubular members 21, 22 to any length along the oral airway member 30. A goal of providing the enclosed tubular flow path of the first and second tubular members 21, 22 to at or near the distal end 32 of the oral airway member 30 is to decrease the amount (e.g., volume or flow rate per unit time) of gas(es) that must be introduced through the oral cannula 10 to provide sufficient gas(es) to the patient where the gas(es) are needed, for example at the posterior pharynx or glottic opening. The use of the hard or rigid oral airway member 30 and/or the providing of this direct, enclosed tubular path through the oral airway member 30 also prevents the patient from obstructing the flow path through the oral cannula 10 prior to the gas(es) reaching the portion of the relevant gas-delivery opening of the patient such as the posterior pharynx or opening of the glottis.

As mentioned above, the oral airway member 30 includes one or more passageways or paths therethrough, such as first passageway 38 and second passageway 39. The passageways 38 and 39 are preferably open, tubular-receptive spaces which may run the length of the oral airway member 30 all the way from the proximal end 31 to the distal end 32. The first tubular member 21 may be disposed (may optionally be completely disposed) within the first passageway 38, while the second tubular member 22 may be disposed (may optionally be completely disposed) within the second passageway 39. The first and second tubular members 21, 22 are preferably connected to the oral airway member 30 within the first passageway 38 and second passageway 39, respectively and may run generally parallel to one another on opposite sides of the one or more connecting members 37. In some embodiments (although not necessarily), the first and second tubular members 21, 22 are completely located and engulfed within their respective passageways 38, 39 so that the tubular members 21, 22 do not protrude past the outer edges of the sides of the oral airway member 30.

FIG. 4 shows the oral cannula 10 disposed within the oral airway passageway of a patient. The oral cannula 10 is disposed in an oral cavity 43 of a patient and may extend from an anterior pharynx region 41, beginning at the mouth entrance, to a posterior pharynx region 42 where the tongue joins with the posterior region of the nasopharyngeal cavity 40. The flared end of the oral cannula (34A, 34B) may abut the patient's lips when the oral cannula 10 is fully inserted into the airway of the patient. The gas may be provided to the patient at or near the upper portion of the trachea 44 via the distal end 32 of the oral cannula 10. In some embodiments, the gas is provided to the patient at an opening of the glottis. The glottis is the opening between two vocal folds. The glottis opens during breathing and closes during swallowing and sound production.

In one embodiment, the oral cannula 10 is formed as one piece and the cannula member 20 and oral airway member 30 are rigidly connected to one another, which may be a permanent connection. In another embodiment, the oral cannula 10 includes a kit having a separate cannula member 20 and oral airway member 30 which are capable of connection to one another for use as the oral cannula 10.

In an alternate embodiment, the oral cannula 10 may include an oral airway member 30 and cannula member 20 connectible to one another in the manner described above, but the cannula member 20 may include a nasal cannula 55 connectible (or already connected to) tubular length extensions 53 and 54. A tubular length extension 53, 54 may be connected to each prong 51, 52 of the nasal cannula 55.

This alternate embodiment allows the oral cannula 10 at least three effective uses in anesthetics. In a first use, when the tubular length extensions 53, 54 are connected to the nasal cannula 55 to form the cannula member 20 and the cannula member 20 is connected to the oral airway member 30, the oral cannula 10 may operate as described above to efficiently deliver one or more gases as well as to maintain the open oral airway passageway through the patient. In a second use, when the tubular length extensions 53, 54 are not connected to the nasal cannula 55 to extend the length of the air delivery prongs 51, 52, the oral airway 10 may be used (although less efficiently and effectively) to deliver supplemental gas(es) to the patient as well as maintain the open oral airway passageway. In a third use, the oral airway member 30 when it is not connected to the nasal cannula 55 or the cannula member 20 may be used only to maintain an open oral airway passageway in the patient, and the nasal cannula 55 may optionally be used to deliver supplemental gas(es) to the nasal passageway of the patient.

The above-described alternate embodiment may include the oral airway member 30, nasal cannula 55, and the tubular length extensions 53, 54 in a three-in-one package or kit. One or more connecting members as known to those skilled in the art may be included with the kit to allow easy and secure connection of the oral airway member 30, nasal cannula 55, and/or tubular length extension 53, 54 to one another as described. The prongs and extensions may be connected to one another using any connecting means or methods known to those skilled in the art for connecting tubular members or cannula to one another. This three-in-one oral cannula kit permits diverse and versatile anesthesia tools to be utilized for a procedure without the need to obtain multiple parts which may or may not fit one another.

The size of the oral cannula 10, particularly the size of the oral airway member 30, may be customized to the patient or may be manufactured and/or sold in a limited amount of sizes, such as for different types of patients (e.g., by weight, adults and children, etc.). Although the cannula member 20 may optionally be of different sizes along with the oral airway member 30, it may be the case that only the oral airway member 30 need vary in size to fit the patient.

It is within the scope of embodiments to substitute the oral airway member 30 with any oral airway or oropharyngeal airway known to those skilled in the art. Similarly, any nasal cannula or other gas-providing cannula known to those skilled in the art may be used in lieu of the cannula member 20 described above.

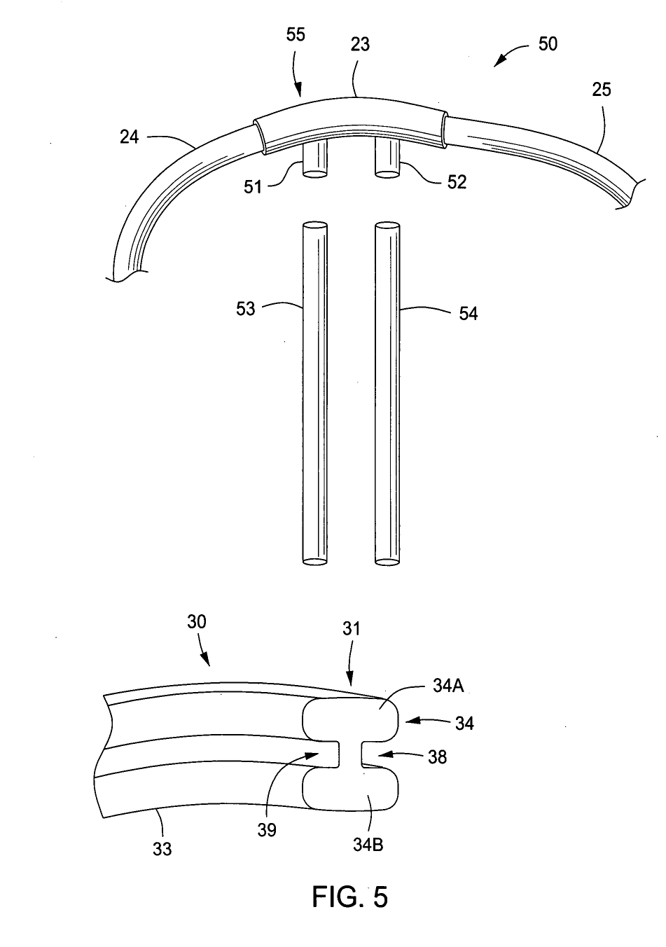

In operation, if the cannula member 20 and oral airway member 30 are not connected to one another originally, the cannula member 20 and oral airway member 30 are connected to one another so that the first tubular member 21 runs along the first passageway 38 through the oral airway member 30 and so that the second tubular member 22 runs along the second passageway 39 through the oral airway member 30. (If the cannula member 20 is not assembled and is in multiple pieces or components, the cannula member 20 is assembled prior to its insertion into and connection to the oral airway member 30. Assembly of the cannula member 20 may involve inserting the first and second tubular members 21, 22 into the third tubular member 23, inserting the tubes 24, 25 into the cannula member 20, and/or inserting the cannula member prong extensions 53, 54 into prongs 51, 52 of a nasal cannula 55 (as in the embodiment shown and described in relation to FIG. 5).)

Once assembly of the oral cannula 10 is accomplished if necessary, ends 26 of the cannula member 20 may be operably connected to one or more gas supply sources such as gas supply tanks (not shown), for example oxygen supply tanks, one or more carbon dioxide monitors, and/or one or more other monitors or supply tanks. This operable connection may be done before or after placement of the oral cannula 10 in the patient's oral airway passageway, which placement is described below.

To place the oral cannula 10 in operable position to create an air passageway between the patient's mouth and the posterior pharynx, the distal end of the oral cannula 10 is inserted over the tongue of the patient. Installing the oral cannula 10 into the patient's oral passageway may be accomplished by any method known to those skilled in the art for inserting an oral or oropharyngeal airway. One method for placing the oral cannula 10 in the patient involves using the tongue blade to depress the tongue, then inserting the distal end 32 of the airway into the patient's throat posteriorly. An alternate method for placing the oral cannula 10 in the patient involves inserting the oral airway upside down until the soft palate is reached, then rotating the device approximately 180 degrees and slipping it over the tongue.

FIG. 4 shows proper placement of the oral cannula 10 in the patient. At the very least, the oral cannula 10 provides an open and unobstructed airway through the oral cannula 10. If desired or necessary, supplemental gas(es) may be provided by introducing one or more gases into the oral cannula 10 via one or more of the gas supply tubes 24, 25 from the gas supply source(s). The one or more gases flow through one or more of the tubes 24, 25 into the cannula member 20, namely from the one or more tubes 24, 25 into the third tubular member 23. Upon exit from the third tubular member 23, the one or more gases flow into the first tubular member 21 and/or second tubular member 22 at or near the proximal end 31 of the oral airway member 30, through the length of the oral airway member 30 via the confined path of the tubular members 21, 22, and out the tubular members 21, 22 at or near the distal end 32 directly into the posterior pharyngeal wall or directly at the opening of the glottis. This direct delivery of the gas(es) allows the supplemental oxygen or other gases to be administered to the patient at much lower flow rates than would be needed if a mere typical nasal cannula is inserted into the oral airway member, because the gas(es) flow along confined paths or passageways (the first and second tubular members 21, 22) to prevent the inefficient and unnecessary escape of these gas(es) from the oral airway member 30.

One or more of the ends 26 of the fluid supply tubes 24, 25 may be operably connected to carbon dioxide monitoring equipment (e.g., a carbon dioxide monitor as is known to those skilled in the art) to measure exhaled carbon dioxide from the patient. In an embodiment, one of the ends is operably connected to an oxygen supply source such as a tank and the other end is operably connected to the carbon dioxide monitoring equipment.

While the patient is under sedation, the patient's vital signs may be monitored throughout the surgical procedure. The oral cannula 10 may be removed at any point during or following the surgical procedure. If desired, the oral cannula 10 may be disassembled and/or disconnected from the gas supply sources and monitoring equipment.

Optionally, the oral cannula may be easily adapted to a breathing circuit for the delivery of anesthetic gases in addition to or in lieu of intravenous agents via operable connection thereto by one or more adapting members. An example adapter capable of use with the oral cannula described above may include a distal diameter of approximately 22 millimeters and a proximal diameter of approximately 6 millimeters (although the adapter dimensions are not limited to these exemplary dimensions).

An estimate of the oxygen flow rate decrease which results from employing the oral cannula described above versus inputting supplemental oxygen at the proximal end of a typical oral airway device (via a typical nasal cannula) is that the flow rate may decrease from approximately 10 L to approximately 2 L flow per unit time, thereby saving 8 L of flow per unit time by providing oxygen (and/or other gases) using the oral cannula described above to provide the oxygen (and/or other gases) at the distal end of the oral airway member of the oral cannula of embodiments. Thus, oxygen supply is advantageously saved using the oral cannula of embodiments.

Some advantages of embodiments described herein, including using an oral cannula in the administration of supplemental oxygen to the sedated patient, are as follows. First, the insertion of the oral cannula into the posterior pharynx of the sedated patient bypasses a large majority of the additional tissue which may be present in an obese or overweight patient and displaces a large tongue which often obstructs the airway in an obese or overweight patient. Second, the extension of oxygen delivery prongs to the distal end of the oral cannula provides supplemental oxygen directly at the opening of the glottis to allow for administration of oxygen at much lower flow rates than would be needed by a typical nasal cannula. Third, the optional incorporation of a carbon dioxide sample tube with one or more connectors, such as one or more male Luer lock connectors, eliminates the need to use separate equipment to detect and monitor adequate ventilation. Fourth, the inclusion of one or more optional adapters would allow easy adaptation of the breathing circuit for the delivery of one or more anesthetic gases in addition to intravenous agents.

While the foregoing is directed to embodiments of the present invention, other and further embodiments of the invention may be devised without departing from the basic scope thereof, and the scope thereof is determined by the claims that follow.

1. An oral cannula apparatus comprising:
an oral airway member comprising:
an enlarged proximal end to prevent the proximal end from slipping down a patient's throat, the proximal end including a first passageway and a second passageway adapted to receive a cannula member therein, the first and second passageways extending along a length of the oral airway member, and
a distal end, the distal end capable of leading the oral airway member upon its insertion into a mouth and pharynx of a patient;
a cannula member having a first cannula and a second cannula extending from a delivery cannula,
wherein the first cannula is located within the first passageway and the second cannula is located within the second passageway, the first and second cannula extending to a location at or near the distal end, and
wherein the first and second cannula provide a delivery path therethrough to provide one or more gases from the first and second cannula directly to an opening of a glottis of the patient.

2. The apparatus of claim 1, wherein the oral airway member is constructed from a rigid material.

3. The apparatus of claim 1, wherein the first cannula is completely engulfed within the first passageway and the second cannula is completely engulfed within the second passageway.

4. The apparatus of claim 1, wherein the first and second cannula provide the one or more gases to the glottis from a location at or near the distal end of the oral airway member.

5. The apparatus of claim 1, wherein the first cannula terminates at location at or near a distal end of the first passageway and the second cannula terminates at a location at or near a distal end of the second passageway.

6. The apparatus of claim 5, wherein the first cannula is operably connected to the oral airway member within the first passageway and the second cannula is operably connected to the oral airway member within the second passageway.

7. The apparatus of claim 1, wherein the first and second cannula extend generally parallel to one another and generally perpendicular to the delivery cannula.

8. The apparatus of claim 1, wherein the delivery cannula is operably connected to a gas supply and provides a flow path for gas from the gas supply to the first and second cannula.

9. The apparatus of claim 1, wherein the gas comprises oxygen.

10. The apparatus of claim 1, wherein the delivery cannula, first cannula, and second cannula comprise a nasal cannula with a first tubular extension member operably connected to the first cannula and a second tubular extension member operably connected to the second cannula to allow the first and second cannula to extend in length to a location at or near the distal end of the oral airway member.

11. The apparatus of claim 1, wherein the delivery cannula, first cannula, and second cannula are formed in one piece.

12. A kit comprising:
a nasal cannula member comprising a delivery cannula and at least two cannula prongs extending generally perpendicularly from the delivery cannula;
an oral airway member; and
at least two cannula extension members, each cannula extension member connectable to each nasal cannula member prong.

13. The kit of claim 12, wherein the kit is interchangeably capable of multi-purpose use, including as a nasal cannula, oral cannula, and extended oral cannula.

14. The kit of claim 12, wherein:
the oral airway member comprises at least two passages formed along its length on opposing sides of the oral airway member; and
an extended oral cannula is formable from the kit by connecting each cannula extension member to each nasal cannula prong and placing each cannula extension member within each passage of the oral airway member.

15. The kit of claim 14, wherein each of the cannula extension members has a length which allows extension of the cannula extension members to a location at or near a distal end of the oral airway member when the cannula extension members are connected to the nasal cannula prongs and placed within the passages of the oral airway member.

16. The kit of claim 15, further comprising one or more connecting members for rigidly connecting the cannula extension members to their respective passages in the oral airway member.

17. A method comprising:
providing an oral cannula comprising:
an oral airway member having an enlarged proximal end and a distal end and first and second passageways extending from the proximal end to the distal end along the length of the oral airway member, and
a cannula member having a first cannula and a second cannula extending generally perpendicularly from a delivery cannula,
wherein the first cannula is located within the first passageway and the second cannula is located within the second passageway and the first and second cannula extend from the proximal end to a location at or near the distal end; and
inserting the distal end of the oral cannula into the mouth and pharynx of a patient until the first and second cannula at the distal end provide direct gas flow access to an opening of a glottis of the patient.

18. The method of claim 17, further comprising delivering one or more gases through the first cannula or second cannula directly to the opening of the glottis.

19. The method of claim 18, wherein the one or more gases comprise oxygen.

20. The method of claim 18, further comprising simultaneously using the oral cannula to provide supplemental oxygen directly to the glottis and maintain an open airway passage in a sedated patient.
